(19) United States
(12) Patent Application Publication
(10) Pub. No.: US 2026/0124033 A1
(43) Pub. Date: May 7, 2026

(54) ARTIFICIAL HEART VALVE

(71) Applicant: BIOTYX MEDICAL (SHENZHEN) CO., LTD., Shenzhen, Guangdong (CN)

(72) Inventors: Gui ZHANG, Shenzhen (CN); Luyao FANG, Shenzhen (CN); Deyuan ZHANG, Shenzhen (CN)

(73) Assignee: BIOTYX MEDICAL (SHENZHEN) CO., LTD., Shenzhen, Guangdong (CN)

(21) Appl. No.: 19/116,507

(22) PCT Filed: Sep. 28, 2023
(86) PCT No.: PCT/CN2023/122612

§ 371 Date: Mar. 28, 2025

(30) Foreign Application Priority Data
Sep. 30, 2022 (CN) 202211206902.1

(51) Int. Cl. A61F 2/24

(52) U.S. Cl. CPC A61F 2/2418; A61F 2210/0061; A61F 2230/0006; A61F 2230/0008; A61F 2230/0019; A61F 2230/0023; A61F 2250/0037; A61F 2250/0069

(57) ABSTRACT

An artificial heart valve includes a stent, a leaflet assembly, an inner skirt, and an outer skirt. The stent includes an inflow end, an outflow end, and stent holes. The outer skirt includes a closing part, a free part, and a connecting part. The closing part is close to the inflow end and connects to the inner skirt. The free part is connected to the closing part and extends away from the inflow end. The connecting part connects to a portion of the free part close to the outflow end and connects to the stent and/or the inner skirt at the same time. Backflow blood can flow through the stent holes located between the outflow end and an inflow edge of the leaflet assembly and flows into the free part. The free part can swell away from the stent under pressure of the backflow blood to abut against surrounding tissues.

### TECHNICAL FIELD

The embodiments belong to the field of medical devices, and to an artificial heart valve.

### BACKGROUND

A heart valve is a one-way valve between an atrium and a ventricle or between a ventricle and an artery. A valvular heart disease is one of the most common cardiovascular diseases. In clinical practice, a single valve structure/multiple valve structures abnormalities caused by rheumatic inflammation, degenerative changes, congenital malformations, ischemic necrosis, trauma, and the like may lead to valvular stenosis or valvular inadequacy. For patients suffering from mild valvular heart diseases, the symptom can be relieved through medication. For patients suffering from severe valvular heart diseases, they need valve repair. If valve repair is not feasible, patients need artificial heart valve replacement.

Due to factors such as calcification of a native valve, vegetations in the native valve, or defects in the native valve, there may be a gap between an artificial heart valve and the native valve after the artificial heart valve replacement. Blood may leak out from the gap between the artificial heart valve and the native valve under a pressure, forming perivalvular leakage. Worsened perivalvular leakage can lead to symptoms such as chest tightness, chest pain, fatigue, and dizziness. Severe perivalvular leakage can lead to consequences such as heart failure and sudden cardiac death.

At present, there have been some methods for preventing perivalvular leakage. For example, an outer skirt with a fibrous edge is added outside an artificial heart valve. The outer skirt promotes blood coagulation in a perivalvular gap, thereby filling a gap between the artificial heart valve and a native valve to achieve the goal of reducing or eliminating perivalvular leakage. However, this method can usually only block small gaps, but has a non-ideal blocking effect on large gaps or notches. For example, there are also methods that use an elastic filling material around an artificial heart valve. A gap between the artificial heart valve and surrounding tissues is reduced by compression and swelling of the filling material, to achieve the goal of reducing and preventing perivalvular leakage. However, a deformation degree of the elastic filling material is limited, so that the blocking effect on large gaps or notches is usually not ideal. It can further easily lead to a further expansion of surrounding tissues, causing damage to the surrounding tissues.

### SUMMARY

To overcome the defects and shortcomings in the existing technology, the embodiments provide an artificial heart valve. An outer skirt of the artificial heart valve can accommodate backflow blood. The blood flows into a free part of the outer skirt from a surface of a leaflet assembly close to the stent via stent holes. Due to non-uniform stress, the blood can flow towards a gap or a notch between surrounding tissues and the stent of the artificial heart valve. Consequently, the free part of the outer skirt can adaptively fill the gap or notch between the surrounding tissues and the stent under the pressure of internal blood, thereby achieving a seal and preventing perivalvular leakage. Meanwhile, as the free part deforms under the pressure of the blood to fill the gap or the notch, the free part does not excessively expand the surrounding tissues, thereby minimizing damage to the surrounding tissues.

The artificial heart valve provided by the embodiments includes:
a stent, including an inflow end and an outflow end, where a plurality of stent holes are formed in the stent;
a leaflet assembly, arranged in the stent, where the leaflet assembly has an inflow edge facing the outflow end;
an inner skirt, arranged on an inner surface of the stent, where the inner skirt includes an inflow skirt edge close to the inflow end and an outflow skirt edge close to the outflow end, and the inner skirt is connected to the inflow edge; and
an outer skirt, at least partially arranged at an outer periphery of the stent, where the outer skirt includes a closing part, a free part, and a connecting part; the closing part is arranged outside the stent and is close to the inflow end; the closing part is connected to the inner skirt; the free part is connected to the closing part and extends in a direction away from the inflow end; the connecting part is connected to a portion of the free part close to the outflow end, and the connecting part is connected to the stent and/or the inner skirt; backflow blood flows through the stent holes located between the outflow end and the inflow edge and flows into the free part; the free part swells in a direction away from the stent under pressure of the backflow blood to abut against surrounding tissues and achieve sealing.

Details of one or more embodiments are set forth in the following drawings and description. Other features, objects, and advantages of the embodiments will become apparent from the drawings and descriptions thereof.

### BRIEF DESCRIPTION OF THE DRAWINGS

To better describe and illustrate the embodiments and/or examples herein, reference may be made to one or more figures. Additional details or examples used to describe the figures should not be considered as limiting the scope of any of the embodiments, the embodiments and/or examples currently described, and the best mode currently understood for embodiments.

FIG. 1a is a three-dimensional diagram of an embodiment of an artificial heart valve according to the embodiments after dilation;

FIG. 1b is an internal detail diagram of the artificial heart valve shown in FIG. 1a;

FIG. 2 is an internal detail diagram of an embodiment of an artificial heart valve according to the embodiments;

FIG. 3 is an internal detail diagram of an embodiment of an artificial heart valve according to the embodiments;

FIG. 4 is an internal detail diagram of an embodiment of an artificial heart valve according to the embodiments;

FIG. 5 is an internal detail diagram of an embodiment of an artificial heart valve according to the embodiments;

FIG. 6 is an internal detail diagram of an embodiment of an artificial heart valve according to the embodiments;

FIG. 7 is an internal detail diagram of an embodiment of an artificial heart valve according to the embodiments;

FIG. 8a is a top view of an embodiment of an artificial heart valve according to the embodiments;

FIG. 8b is a top view of an embodiment of an artificial heart valve according to the embodiments;

FIG. 8c is a top view of an embodiment of an artificial heart valve according to the embodiments;

FIG. 8d is a top view of an embodiment of an artificial heart valve according to the embodiments;

FIG. 9a is a schematic diagram of preventing perivalvular leakage after an artificial heart valve is implanted into a human body according to the embodiments;

FIG. 9b is an enlarged view of part A in FIG. 9a; and

FIG. 10a is a cross-sectional view of an embodiment of an artificial heart valve according to the embodiments;

FIG. 10b is a cross-sectional view of an embodiment of an artificial heart valve according to the embodiments;

FIG. 10c is a cross-sectional view of an embodiment of an artificial heart valve according to the embodiments;

FIG. 10d is a cross-sectional view of an embodiment of an artificial heart valve according to the embodiments;

### DETAILED DESCRIPTION OF THE EMBODIMENTS

The exemplary implementations of the embodiments will be described in more detail below with reference to the accompanying drawings. Although the accompanying drawings show the exemplary implementations of the embodiments, it should be understood that the embodiments can be implemented in various forms, and should not be limited to the implementations stated herein. Rather, these implementations are provided for understanding the embodiments more thoroughly, and can completely transfer the scope of the embodiments to those skilled in the art.

An artificial heart valve provided by the embodiments is suitable for being implanted into a natural or artificial arterial valve, such as an aortic valve, a mitral valve, a tricuspid valve, and a pulmonary artery valve. The artificial heart valve can be implanted into a human body through percutaneous minimally invasive intervention guided by imaging equipment such as X-ray and/or ultrasound, or through surgical thoracotomy.

In some embodiments, the artificial heart valve may include a stent, a leaflet assembly, and an inner skirt. Referring to an anterograde direction of a blood flow, one end where an anterograde blood flow flows into the stent can be an inflow end, and one end where the anterograde blood flow flows out of the stent can be an outflow end. One side edge of the leaflet assembly facing the inflow end can be connected to the stent through an inner skirt. After the artificial heart valve is implanted into a human body, when the ventricles contract, anterograde blood flows into the inflow end of the stent. The anterograde blood compresses the leaflet assembly, and the leaflet assembly is opened and abuts against an inner surface of the stent. The anterograde blood flows through the leaflet assembly to the outflow end. When the ventricles begin to dilate, no blood flows into the inflow end, and the pressure at the outflow end is greater than that at the inflow end. The pressure forces the blood at the outflow end to flow back towards the inflow end, and the leaflet assembly is pushed to be gathered and closed by the backflow blood, to prevent the blood from flowing back. Therefore, the artificial heart valve has the functions of a natural heart valve, and can control the blood to flow in only one direction from the inflow end to the outflow end, without backflow.

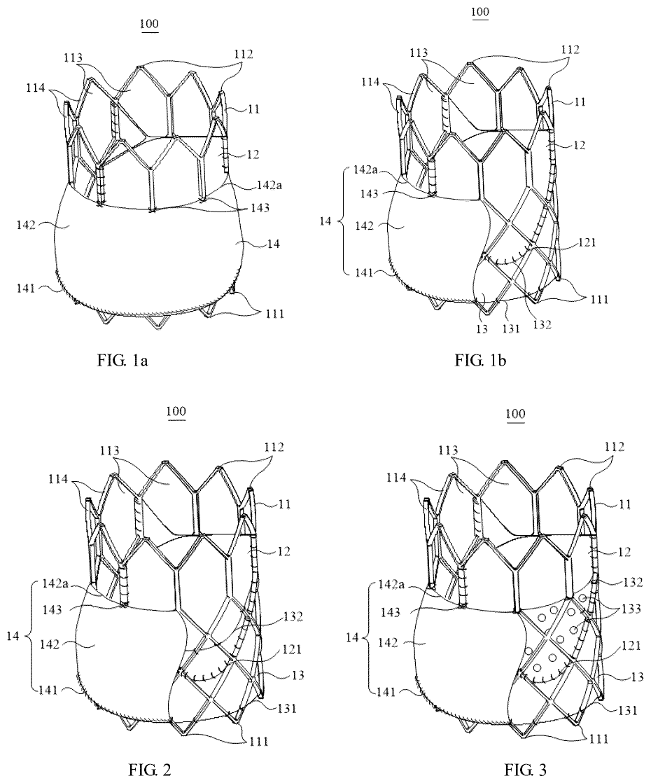

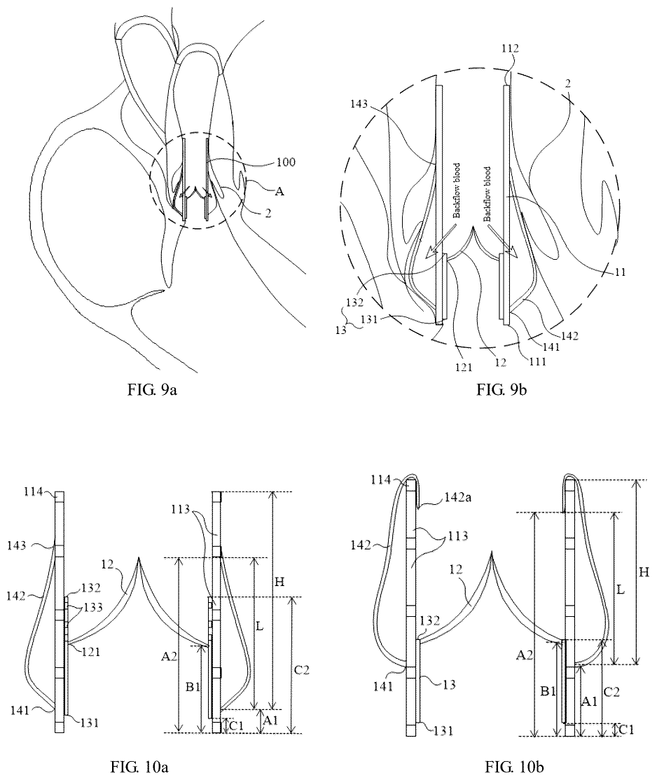

Referring to FIG. 1a to FIG. 10d, an artificial heart valve 100 provided by the embodiments may include a stent 11, a leaflet assembly 12, an inner skirt 13, and an outer skirt 14. The stent 11 may include an inflow end 111 and an outflow end 112. A plurality of stent holes 113 may be formed in the stent 11. The leaflet assembly 12 may be arranged in the stent 11. The leaflet assembly 12 may have an inflow edge 121 facing the outflow end 112. As an example, the inflow edge 121 is located on a surface of the leaflet assembly 12 facing away from an axial center of the stent 11 (i.e., a surface facing the outside of the stent), which may be a position where the leaflet assembly 12 is connected to the inner skirt 13 or the stent 11. The backflow blood flowing back to the surface of the leaflet assembly 12 may at least flow from a stent hole 113 close to the inflow edge 121 towards a free part 142. The inner skirt 13 may be arranged on an inner surface of the stent 11. The inner skirt 13 may include an inflow skirt edge 131 close to the inflow end 111 and an outflow skirt edge 132 close to the outflow end 112. The inflow skirt edge 131 is close to the inflow end 111, and the outflow skirt edge 132 is close to the outflow end 112 or closer to the outflow end 112 than the inflow skirt edge 131. The inner skirt 13 may be connected to the inflow edge 121. The outer skirt 14 may be at least partially arranged at an outer periphery of the stent 11. The outer skirt 14 may include a closing part 141, a free part 142, and a connecting part 143. The closing part 141 may be arranged outside the stent 11, and the closing part 141 may be arranged close to the inflow end 111. The closing part 141 may be connected to the inner skirt 13. The free part 142 may be connected to the closing part 141 and may extend in a direction away from the inflow end 111. The connecting part 143 may be connected to a portion of the free part 142 close to the outflow end 112, and the connecting part 143 may be connected to the stent 11 and/or the inner skirt 13. Backflow blood can flow through the stent holes 113 located between the outflow end 112 and the inflow edge 121 and flow into the free part 142. The free part 142 can swell in a direction away from the stent 11 under pressure of the backflow blood to abut against surrounding tissues 2 and achieve sealing.

The stent 11 can open up the narrow or occluded surrounding tissues 2 and provide support and fixation for the leaflet assembly 12, the inner skirt 13, and the outer skirt 14. The leaflet assembly 12 can be opened or closed to control the flow of the blood. The inner skirt 13 can hermetically connect the leaflet assembly 12 to the stent 11 by connecting the inflow edge 121 of the leaflet assembly 12. The closing part 141 seals one side of the free part 142 close to the inflow end 111, so that blood in the free part 142 cannot leak out from the side of the free part 142 close to the inflow end 111. The connecting part 143 is connected to a portion of the free part 142 close to the outflow end 112, and the flexible free part 142 is fixed to the stent 11, without prolapse from the stent 11, so that the blood in the free part 142 cannot leak out from the side of the free part 142 close to the outflow end 112. The backflow blood flows from a surface of the leaflet assembly 12 to the stent holes 113 provided between the outflow end 112 and the inflow edge 121, and further flows into the free part 142 of the outer skirt 14. When there is a gap or notch between the stent 11 and the surrounding tissues 2, the blood may flow towards the gap or the notch due to non-uniform stress, thereby pushing the flexible free part 142 to swell towards the gap or notch. Therefore, the free part 142 adaptively fills the gap or the notch between the stent 11 and the surrounding tissues 2 under the pushing of the blood and achieves sealing, thereby achieving an effect of preventing perivalvular leakage on surrounding tissues 2 with different shapes. Meanwhile, the free part 142 may not cause significant compression on the surrounding tissues 2 when tightly abutting against the surrounding tissues 2, and may not further expand the surrounding tissues 2, so that damage to the surrounding tissues 2 is small.

In the artificial heart valve 100, the stent 11 may have both a dilated state and a contracted state. The stent 11 may be dilated by a dilation device (such as a dilation balloon) or may self-expand. The stent 11 may be made of a biologically absorbable material. For example, the stent 11 may be made of iron, an iron-based alloy, magnesium, a magnesium-based alloy, zinc, a zinc-based alloy, or an absorbable polymer material. Thus, after the valve is implanted into the human body for endothelialization, the stent 11 can be absorbed by the body, thereby eliminating rigid pulling on leaflets, and can have a good postoperative recovery effect, buffer the tension during closing of the leaflets, and prolong the fatigue time of the leaflets. Secondly, after the stent 11 is absorbed, valve-in-valve replacement of the artificial valve may not be affected, ensuring that a valve opening area does not decrease. The stent 11 may be made of a biologically non-absorbable material. For example, the stent 11 may be made of a material such as a nickel titanium alloy, a cobalt chromium alloy, or stainless steel.

In the artificial heart valve 100, as described above, the inflow end 111 of the stent 11 may be an end where anterograde blood flows into the stent 11. The outflow end 112 of the stent 11 may be an end where the anterograde blood flows out of the stent 11. The stent 11 may further include a middle part (not shown) located between the inflow end 111 and the outflow end 112. Nominal diameters of the inflow end 111, the outflow end 112, and the middle part may be the same or different. The stent 11 may include a plurality of stent rods 114. The plurality of stent rods 114 may be arranged in a crossed manner to form the plurality of stent holes 113. Sizes and shapes of the stent holes 113 may change as a dilation degree of the stent 11 changes. The stent holes 113 may be roughly diamond-shaped, hexagonal, triangular, irregularly-shaped, or the like.

In the artificial heart valve 100, the leaflet assembly 12 may include at least two leaflets, for example, two or three leaflets. At least two leaflets may be arranged in the stent 11 in a circumferential direction of the stent 11. The leaflets can be set according to a shape of a natural leaflet, for example, roughly like a sector. The inflow edge 121 of the leaflet assembly 12 may be circular-arc-shaped. The leaflets may be made from a natural biological tissue, such as decellularized bovine pericardium, porcine pericardium, porcine aortic valve, fish maw, or a small intestine tissue of a mammal.

In the artificial heart valve 100, the inner skirt 13 may be arranged on an inner surface of the stent 11, and the inner skirt 13 may be connected to the stent 11. The inner skirt 13 may be hermetically connected to the inflow edge 121 of the leaflet assembly 12 to fix the leaflet assembly 12 in the stent 11. The inner skirt 13 and the stent 11, as well as the inner skirt 13 and the inflow edge 121, may be sutured, glued, and/or welded, respectively. Spread shapes of the inflow skirt edge 131 and the outflow skirt edge 132 may be like a straight line, a curved line, a broken line, or the like.

In the artificial heart valve 100, the outer skirt 14 may be at least partially arranged at an outer periphery of the stent 11. In other words, the outer skirt 14 may be located at the outer periphery of the stent 11. Or, an extending end 142a of the outer skirt 14 close to the outflow end 112 may extend out of the outflow end 112 and be folded back into the stent 11, so that a portion of the outer skirt 14 is located at the outer periphery of the stent 11, and the remaining portion is located inside the stent 11.

The closing part 141 of the outer skirt 14 may extend outside the stent 11 in the circumferential direction of the stent 11. The closing part 141 may be arranged close to the inflow end 111, namely, the closing part 141 may be located between the middle part of the stent 11 and the inflow end 111 of the stent 11. The closing part 141 may be connected to both the inner skirt 13 and the stent 11, thereby enhancing the fixing effect of the closing part 141 on the stent 11. The closing part 141 and the inner skirt 13, as well as the closing part 141 and the stent 11 may be sutured, glued, and/or welded too.

The free part 142 and the closing part 141 of the outer skirt 14 may be integrally or separately formed, and the free part 142 and the closing part 141 may be sutured, glued, and/or welded. The free part 142 may extend in the circumferential direction and an axial direction of the stent 11. The extending end 142a of the free part 142 may be located outside the stent 11, or may extend out of the outflow end 112 and be folded back into the stent 11.

The connecting part 143 of the outer skirt 14 may be connected to a portion of the free part 142 close to the outflow end 112. For example, the connecting part 143 may be connected to a portion of the free part 142 close to the extending end 142a, or may be connected to the extending end 142a. The connecting part 143 and the free part 142 may be integrally formed or detachably connected. For example, they are sutured, glued, and/or welded.

In the artificial heart valve 100, the backflow blood can flow through the stent holes 113 located between the outflow end 112 and the inflow edge 121 and flow into the free part 142. In other words, the stent holes 113 between the outflow end 112 and the inflow edge 121 may be at least partially exposed, or the outer skirt 14 (if any) and/or the inner skirt 13 (if any) covering the stent holes 113 inside the stent 11 may allow blood to pass or permeate through. It can be understood that the shape of the inflow edge 121 and the shape of each stent hole 113 are generally not the same. For example, a trajectory of the inflow edge 121 does not overlap with edges of the stent hole 113, or is blocked by the outer skirt 14 (if any) and/or the inner skirt 13 (if any), and the backflow blood may pass through an entire or partial hole region of the plurality of stent holes 113 mentioned above.

The following provides a detailed introduction to the artificial heart valve 100 of the embodiments.

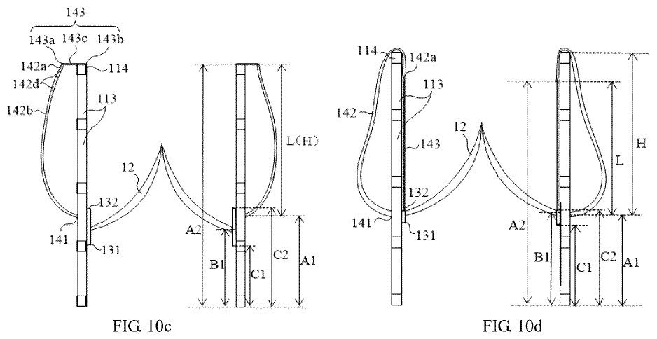

Referring to FIG. 10a to FIG. 10d, an axial distance between the closing part 141 and the inflow end 111 may be A1. A minimum axial distance between the inflow edge 121 and the inflow end 111 may be B1. An axial distance between the inflow skirt edge 131 and the inflow end 111 may be C1. An axial distance between the outflow skirt edge 132 and the inflow end 111 may be C2. In the artificial heart valve 100 of the embodiments, a relative position between the closing part 141 and the inflow edge 121 may be set. That is, A1 maybe greater than, equal to, or less than B1, so that the free part 142 can adapt to surrounding tissues 2 with different shapes at different implantation positions to achieve an effect of preventing perivalvular leakage.

Referring to FIG. 10a and FIG. 10b, in some embodiments, A1<B1, C1<A1, C2>B1. Namely, the axial distance A1 between the closing part 141 and the inflow end 111 may be less than the minimum axial distance B1 between the inflow edge 121 and the inflow end 111. Since the inner skirt 13 is connected to the closing part 141, the axial distance C1 between the inflow skirt edge 131 and the inflow end 111 may be less than (slightly or significantly less) than the axial distance A1 between the closing part 141 and the inflow end 111. Since the inner skirt 13 may be connected to the inflow edge 121, the axial distance C2 between the outflow skirt edge 132 and the inflow end 111 may be greater (slightly or significantly greater) than the minimum axial distance B1 between the inflow edge 121 and the inflow end 111. In the above embodiments, the closing part 141 may be closer to the inflow end 111 than the inflow edge 121 (a portion closest to the inflow end 111), so that the free part 142 can be better suitable for sealing a gap or notch close to the inflow end 111. In addition, the inner skirt 13 is connected to the inflow edge 121; the axial distance A1 between the closing part 141 and the inflow end 111 is less than the minimum axial distance B1 between the inflow edge 121 and the inflow end 111; and the axial distance C1 between the inflow skirt edge 131 and the inflow end 111 is less than the axial distance A1 between the closing part 141 and the inflow end 111. Thus, the stent holes 113 located between the closing part 141 and the inflow edge 121 are covered or closed by the inner skirt 13 from the inner side. On the one hand, a closed “pocket” facing the outflow end 112 is formed on a backflow blood path, thereby achieving an effect of preventing perivalvular leakage.

Referring to FIG. 10d, in another embodiment, A1=B1, C1<A1, C2>B1. Namely, the axial distance A1 between the closing part 141 and the inflow end 111 may be equal to the minimum axial distance B1 between the inflow edge 121 and the inflow end 111. The axial distance C1 between the inflow skirt edge 131 and the inflow end 111 may be less (slightly or significantly less) than the axial distance A1 between the closing part 141/the inflow edge 121 and the inflow end 111, and the axial distance C2 between the outflow skirt edge 132 and the inflow end 111 may be greater (slightly or significantly greater) than the minimum axial distance B1 between the inflow edge 121/or the closing part 141 and the inflow end 111. In the above embodiment, the closing part 141 is flush with a portion of the inflow edge 121 closest to the inflow end 111, and the free part 142 can be better suitable for sealing a gap or notch approximately in a middle part of the stent 11.

Referring to FIG. 10c, in still another embodiment, A1>B1, C1<B1, C2>A1. Namely, the axial distance A1 between the closing part 141 and the inflow end 111 may be greater than the minimum axial distance B1 between the inflow edge 121 and the inflow end 111. The axial distance C1 between the inflow skirt edge 131 and the inflow end 111 may be less (slightly or significantly less) than the minimum axial distance B1 between the inflow edge 121 and the inflow end 111. The axial distance C2 between the outflow skirt edge 132 and the inflow end 111 may be greater (slightly or significantly greater) than the axial distance A1 between the closing part 141 and the inflow end 111. In the above embodiments, the closing part 141 may be closer to the outflow end 112 than the inflow edge 121 (a portion closest to the inflow end 111), so that the free part 142 can be better suitable for sealing a gap or notch close to the outflow end 112.

The above embodiments can be compared on the same axial cross section. This axial cross section may pass through the portion of the inflow edge 121 closest to the inflow end 111, namely, the minimum axial distance between the inflow edge 121 and the inflow end 111 may be an axial distance between the portion of the inflow edge 121 closest to the inflow end 111 and the inflow end 111.

Further, when A1≤B1, at least a portion of the inner skirt 13 located between the inflow edge 121 and the outflow skirt edge 132 is permeable to the backflow blood. When A1>B1, at least a portion of the inner skirt 13 located between the closing part 141 and the outflow skirt edge 132 is permeable to blood. The portion of the inner skirt 13 between the inflow edge 121/the closing part 141 and the outflow skirt edge 132 can hinder the backflow blood from entering the free part 142. By setting this portion of the inner skirt 13 to be at least partially permeable to blood, more backflow blood can enter the free part 142 to achieve a better effect of preventing perivalvular leakage. The blood-permeable portion of the inner skirt 13 may be made from a blood-permeable material. Referring to FIG. 3, FIG. 6, and FIG. 10a, the blood-permeable portion of the inner skirt 13 may be provided with a via hole 133 that allows blood to permeate through. The via hole 133 may be triangular, circular, square, polygonal, waist-shaped, irregularly shaped, or the like. One or at least two via holes 133 may be provided. The at least two via holes 133 may be spaced apart or connected in a circumferential direction of the inner skirt 13.

In addition, C1 (the axial distance between the inflow skirt edge 131 and the inflow end 111) may be greater than 0 and less than or equal to 40% of a nominal length of the stent 11. Therefore, on the premise of ensuring a good connection effect on the inner skirt 13 and the stent 11, the exposed inflow end 111 can be embedded into the surrounding tissues 2, and the stent 11 can be limited by the surrounding tissues 2, thereby enhancing the fixation effect of the artificial heart valve 100 in the human body. Further, C1 may be 5% to 40%, 5% to 35%, 8% to 35%, 10% to 30%, 15% to 20%, or 10% to 15% of the nominal length of the stent 11. For example, C1 may be 1%, 5%, 8%, 10%, 15%, 20%, 25%, 30%, 35%, or 40% of the nominal length of the stent 11. The above data may be C1 at any axial cross section, or an average value of C1s at a plurality of arbitrary axial cross sections.

Generally, C1 may be greater than or equal to 0.1 mm and less than or equal to 24 mm. Further, C1 may be greater than or equal to 0.5 mm and less than or equal to 20 mm. Still further, C1 may be greater than or equal to 1 mm and less than or equal to 15 mm. C1 can include, but is not limited to, 0.1 mm, 0.5 mm, 1 mm, 3 mm, 5 mm, 10 mm, 15 mm, 20 mm, or 24 mm. For the above list, refer to the artificial heart valve 100 with a conventional nominal length (such as 15 to 60 mm) only, which will not be limited to this.

In the artificial heart valve 100 of the embodiments, the free part 142 and the closing part 141 can be spaced apart from the inflow end 111 of the stent 11. A1 (the axial distance between the closing part 141 and the inflow end 111) may be greater than or equal to 1% of the nominal length of the stent 11 and less than or equal to 49% of the nominal length of the stent 11. By adjusting the distance between the closing part 141 and the inflow end 111, the relative position between the free part 142 and the stent 11 can be adjusted to an extent. When A1 is less than 1% of the nominal length of the stent 11, an extending length of the free part 142 between the closing part 141 and the inflow edge 121 is relatively large, and a curled contour is relatively large, which increases the difficulty of percutaneous minimally invasive intervention. When A1 is greater than 49% of the nominal length of the stent 11, a contact area between the free part 142 and surrounding tissues 2 is insufficient, which may reduce the effect of preventing perivalvular leakage. A1 may be consistent or inconsistent on different axial cross sections. The above data may be A1 at any axial cross section, or an average value of Als at a plurality of arbitrary axial cross sections.

Further, A1 may be 3% to 49%, 5% to 40%, 8% to 30%, 10% to 40%, 5% to 35%, 15% to 30%, 15% to 20%, 25% to 30%, or the like of the nominal length of the stent 11. For example, A1 may be 1%, 2%, 5%, 8%, 10%, 15%, 20%, 25%, 30%, 35%, 40%, 45%, and 49% of the nominal length of the stent 11, or any value between the above data.

As a reference, A1 may be greater than or equal to 0.3 mm and less than or equal to 28 mm. Further, A1 may be greater than or equal to 0.5 mm and less than or equal to 25 mm. Still further, A1 may be greater than or equal to 1 mm and less than or equal to 20 mm. For example, A1 may be 0.3 mm, 0.5 mm, 1 mm, 3 mm, 4 mm, 5 mm, 7 mm, 8 mm, 10 mm, 15 mm, 20 mm, 25 mm, 28 mm, or any distance between the above data. It should be noted that the above data range may be suitable for the artificial heart valve 100 with the conventional nominal length, and may be adjusted appropriately for an artificial heart valve 100 with a special size.

Referring to FIG. 10a to FIG. 10d, an axial distance between the extending end 142a of the free part 142 after swelling and the inflow end 111 may be A2. A difference between A2 and B1 (the minimum axial distance between the inflow edge 121 and the inflow end 111) may be greater than or equal to 30% of the nominal length of the stent 11 and less than or equal to 74% of the nominal length of the stent 11. Therefore, the free part 142 has a sufficient space to accommodate the backflow blood, and the blood may not leak out from the extending end 142a of the free part 142, thus achieving an effect of preventing perivalvular leakage.

Further, the difference between A2 and B1 may be 35% to 74%, 35% to 70%, 40% to 70%, 45% to 60%, 35% to 70%, 50% to 70%, 60% to 74%, 55% to 65%, or 45% to 60% of the nominal length of the stent 11. Generally, the difference between A2 and B1 may be greater than or equal to 5 mm and less than or equal to 40 mm. Further, the difference between A2 and B1 may be greater than or equal to 6 mm and less than or equal to 35 mm. Still further, the difference between A2 and B1 may be greater than or equal to 7 mm and less than or equal to 30 mm. For example, the difference between A2 and B1 may be 5 mm, 6 mm, 7 mm, 8 mm, 10 mm, 20 mm, 25 mm, 30 m m, 35 mm, 39 mm, 40 mm, or any length between adjacent values mentioned above. Similarly, for the above data, refer to the nominal length of the conventional stent 11 only, which will not be limited to this.

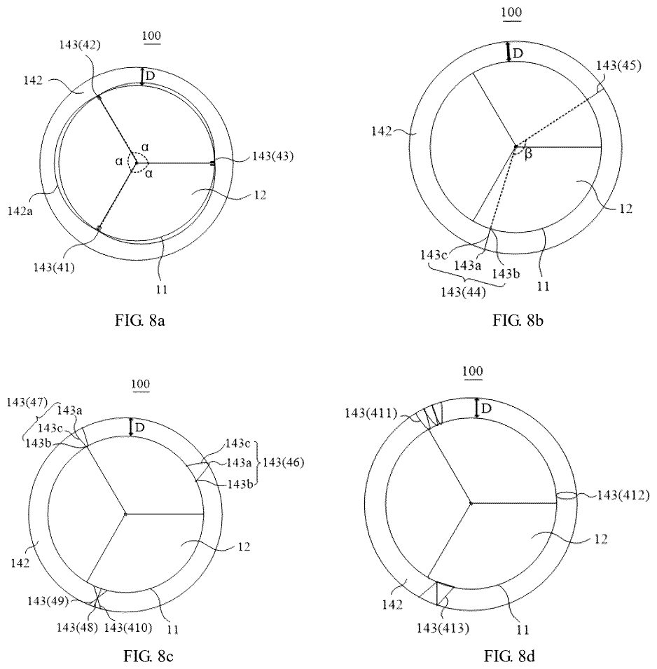

The free part 142 has a radial extension after swelling. Referring to FIG. 8a to FIG. 8d, the radial extension (represented by D in the figure) of the free part 142 after swelling may be defined as a distance between an outer surface of the free part 142 and an outer surface of the stent 11 on a radial cross section. The radial extension of the free part 142 after swelling may be related to a nominal diameter and/or nominal length of the stent 11 when the stent 11 is spread, namely, the radial extension of the free part 142 after swelling can vary on stents 11 with different nominal diameters and/or nominal lengths. Further, the radial extension of the free part 142 after swelling may be the same on stents 11 with different nominal diameters and/or nominal lengths.

The radial extension D of the free part 142 after swelling may be greater than or equal to 5% of the nominal diameter of the stent 11 and less than or equal to half of the nominal length of the stent 11. Thus, the swelling outer skirt 14 has a sufficient radial expansion amplitude, which can adapt well to fill gaps or notches with different sizes between the stent 11 and the surrounding tissues 2, thus achieving a good effect of preventing perivalvular leakage. Moreover, the artificial heart valve 100 has a relatively small overall curled contour under a radially compressed state. When D is less than 5% of the nominal diameter of the stent 11, the free part 142 may not be able to seal a relatively large gap or notch. When D is greater than half of the nominal length of the stent 11, the curled contour of the free part 142 in the radially compressed state is relatively large, which increases the difficulty of percutaneous minimally invasive intervention. Similarly, the above data range may be D at any radial cross section, or an average value of Ds at a plurality of arbitrary radial cross sections.

Further, D may be greater than or equal to 8% of the nominal diameter of the stent 11 and less than or equal to 50% of the nominal length of the stent 11. Or, D may be greater than or equal to 10% of the nominal diameter of the stent 11 and less than or equal to 45% of the nominal length of the stent 11. Or, D may be greater than or equal to 9% of the nominal diameter of the stent 11 and less than or equal to 30% of the nominal length of the stent 11. Or, D may be greater than or equal to 12% of the nominal diameter of the stent 11 and less than or equal to 40% of the nominal length of the stent 11. Generally, D may be greater than or equal to 0.8 mm and less than or equal to 25 mm. Further, D may be 1 to 23 mm, 2 to 25 mm, 3 to 20 mm, 5 to 10 mm, 5 to 15 mm, 5 to 18 mm, 8 to 13 mm, 10 to 18 mm, 16 to 20 mm, or the like. D may include, but is not limited to, 0.8 mm, 1 mm, 2 mm, 3 mm, 5 mm, 8 mm, 10 mm, 13 mm, 15 mm, 18 mm, 20 mm, or 25 mm. Of course, if the nominal diameter and/or nominal length of the stent 11 exceed a conventional range, D may not be limited to the above data ranges.

Referring to FIG. 10a to FIG. 10d, the axial distance between the extending end 142a of the free part 142 after swelling and the closing part 141 may be L; the axial distance between the outflow end 112 and the closing part 141 may be H; and a length of the free part 142 may be greater than or equal to L and less than or equal to (H−1/2L)π. The value of L may be equal to A2-A1. H−1/2L may be represented as H-½L, or H−1/2 (A2−A1). Therefore, the free part 142 has a relatively appropriate extending length, and the free part 142 and the surrounding tissues 2 have a relatively appropriate contact area, thus achieving a good effect of preventing perivalvular leakage.

Referring to FIG. 10a and FIG. 10c, wen the extending end 142a of the free part 142 is located outside the stent 11, the length of the free part 142 may be greater than or equal to L and less than or equal to ½Lπ. Referring to FIG. 10b and FIG. 10d, when the extending end 142a extends out of the outflow end 112 and is folded back into the stent 11, the length of the free part 142 may be greater than or equal to H+L and less than or equal to (H−1/2L)π.

Generally, the length of the free part 142 may be greater than or equal to 10 mm and less than or equal to 31 mm. Further, the length of the free part 142 may be 10 to 30 mm, 13 to 25 mm, 12 to 20 mm, 15 to 28 mm, 15.7 mm to 20 mm, 18 to 25 mm, or the like. For example, the length of the free part 142 may include, but is not limited to, 10 mm, 10.5 mm, 12 mm, 13 mm, 15 mm, 15.7 mm, 16 mm, 16.5 mm, 17 mm, 18 mm, 19 mm, 20 mm, 25 mm, 28 mm, 30 mm, or 31 mm. Similarly, the above data ranges may be suitable for an artificial heart valve 100 with a conventional nominal length, and may be adjusted correspondingly for an artificial heart valve 100 with a special size.

A thickness of the free part 142 is greater than or equal to 5 μm and less than or equal to 200 μm. Further, the thickness of the free part 142 may be greater than or equal to 10 μm and less than or equal to 190 μm. Further, the thickness of the free part 142 may be greater than or equal to 20 μm and less than or equal to 180 μm. Therefore, the free part 142 can have a relatively small curled contour while having a good effect of preventing perivalvular leakage. When the thickness of the free part 142 is less than 5 μm, the free part 142 is easily torn during spreading of the stent 11. When the thickness of the free part 142 is greater than 200 μm, the curled contour of the free part 142 is relatively large, which increases the difficulty of performing percutaneous minimally invasive intervention on the artificial heart valve 100. The thickness of the free part 142 may be overall consistent or may vary in the axial direction and/or the radial direction. The above data may be a thickness at any radial cross section, or an average thickness of thicknesses at a plurality of arbitrary radial cross sections. For example, the thickness of the free part 142 may be, but is not limited to, 5 μm, 7 μm, 10 μm, 15 μm, 18 μm, 20 μm, 30 μm, 50 μm, 100 μm, 150 μm, 180 μm, 190 μm, or 200 μm.

Referring to FIG. 1a to FIG. 10d, as mentioned above, the outer skirt 14 may include a connecting part 143. Referring to FIG. 1a to FIG. 8a and FIG. 9a to FIG. 10b, in some embodiments, the connecting part 143 may include a first connecting sub-part 143a and a second connecting sub-part 143b which are connected to each other. One side or one end of the first connecting sub-part 143a away from the second connecting sub-part 143b may be connected to the free part 142, and one side or one end of the second connecting sub-part 143b away from the first connecting sub-part 143a may be connected to the stent 11 and/or the inner skirt 13. The first connecting sub-part 143a and the second connecting sub-part 143b may be two opposite sides or two adjacent sides of the connecting part 143. Through the first connecting sub-part 143a and the second connecting sub-part 143b, the free part 142 may be tightly connected to the outside of the stent 11 by the connecting part 143, and the connection between the free part 142 and the stent 11 and/or the inner skirt 13 is relatively stable. The first connecting sub-part 143a and the second connecting sub-part 143b may be ringlike, strip-shaped, block-shaped, point-like, or the like. The shape of the first connecting sub-part 143a and the shape of the second connecting sub-part 143b may be the same or different.

Referring to FIG. 8b to FIGS. 8d, 10c, and 10d, in some other embodiments, the connecting part 143 includes a first connecting sub-part 143a, a second connecting sub-part 143b, and an extending sub-parts 143c, and two ends of the extending sub-part 143c are respectively connected to the first connecting sub-part 143a and the second connecting sub-part 143b. One side or one end of the first connecting sub-part 143a away from the extending sub-part 143c may be connected to the free part 142, and one side or one end of the second connecting sub-part 143b away from the extending sub-part 143c may be connected to the stent 11 and/or the inner skirt 13. The first connecting sub-part 143a and the extending sub-part 143c, as well as the second connecting sub-part 143b and the extending sub-part 143c, may be sutured, glued, and/or welded, respectively. Due to the arrangement of the extending sub-part 143c the free part 142 can be relatively loosely connected to the outside of the stent 11, thereby increasing the radial extension of the free part 142 after swelling and providing an axial margin for the free part 142 with the contraction of the stent 11. The extending sub-part 143c may be strip-shaped, rod-shaped, sheet-like, or the like.

At least one connecting part 143 may be provided, namely, there may be one or more connecting parts 143. To achieve a better effect of preventing perivalvular leakage, the number of the connecting part 143 may be related to the shape of the first connecting sub-part 143a (as described in detail later). In the same connecting part 143, there may be at least one first connecting sub-part 143a. Also, in the same connecting part 143, there may be at least one second connecting sub-parts 143b and/or at least one extending sub-parts 143c (if any).

For example, when more than one first connecting sub-parts 143a, more than one second connecting sub-parts 143b, and/or more than one extending sub-parts 143c (if any) are provided, the first connecting sub-parts 143a of the same or different connecting parts 143 may be spaced apart or connected to each other (such as crossing or at least partially overlapping) on the free part 142. The second connecting sub-parts 143b of the same or different connecting parts 143 may be spaced part or connected to each other (such as crossing or at least partially overlapping) on the stent 11 and/or inner skirt 13. The extending sub-parts 143c (if any) of the same or different connecting parts 143 may be spaced apart or connected to each other (such as crossing or at least partially overlapping) between the stent 11 and the free part 142.

The embodiments are not limited to this. For example, the first connecting sub-parts 143a may alternatively be connected to the second connecting sub-parts 143b and/or the extending sub-parts 143c (if any) of other connecting parts 143. The second connecting sub-parts 143b may alternatively be connected to the first connecting sub-parts 143a and/or the extending sub-parts 143c (if any) of the same or other connecting parts 143. The extending sub-parts 143c (if any) may alternatively be respectively connected to the first connecting sub-parts 143a and/or the second connecting sub-parts 143b of the same or other connecting parts 143.

Further, in some embodiments, at least one first connecting sub-part 143a and at least one second connecting sub-part 143b are provided, and at least two extending sub-parts 143c are provided. Two ends or two sides of any extending sub-part 143c are respectively connected to the first connecting sub-part 143a and the second connecting sub-part 143b, and one end or one side of each remaining extending sub-part 143c have is connected to the first connecting sub-part 143a or the second connecting sub-part 143b, and the other end or the other side is connected to the first connecting sub-part 143a or the second connecting sub-part 143b.

An axial distance between at least one first connecting sub-part 143a and an extending end 142a of the free part 142 may greater than or equal to 0 mm and less than or equal to 10 mm. Thus, the extending end 142a of the free part 142 or a portion of the free part 142 close to the extending end 142a may be connected to the stent 11 by the first connecting sub-parts 143a, which can increase the effective utilization rate of the free part 142. The free part 142 has a relatively large space to accommodate the backflow blood. When the axial distance between the first connecting sub-part 143a and the extending end 142a is greater than 10 mm, the free part 142 has a relatively small accommodating space, and the effective utilization rate is relatively low.

Further, the axial distance between at least one first connecting sub-part 143a and the extending end 142a may be 0 to 9 mm, 0 to 8 mm, 1 to 9 mm, 0 to 7 mm, 1 to 6 mm, 2 to 5 mm, 2 to 4 mm, or the like. For example, the axial distance between at least one of the first connecting sub-parts 143a and the extending end portion 142a may include but is not limited to 0 mm, 0.5 mm, 1 mm, 1.5 mm, 2 mm, 4 mm, 6 mm, 8 mm, 8.5 mm, 9 mm, or 10 mm.

In some embodiments, at least one first connecting sub-parts 143a may be strip-shaped or ringlike (as shown in FIG. 6) from a top-view surface. By taking a center of the stent 11 as a vertex, an angle between two ends of the strip-shaped first connecting sub-part 143a from the top-view surface is greater than or equal to 120° and less than 360°. Further, the above angle may be greater than or equal to 130° and less than or equal to 350°. Still further, the above angle may be greater than or equal to 140° and less than or equal to 340°. For example, the above angle may be 120°, 130°, 140°, 180°, 220°, 260°, 300°, 320°, 330°, 340°, 350°, or any range between the above values. It should be noted that the above top-view surface may be a set of a plurality of radial cross sections, namely, the shape of the first connecting sub-part 143a from the top-view surface may be a shape formed by the plurality of radial cross sections overlapping together.

In some other embodiments, at least two first connecting sub-parts 143a may be point-like from the top-view surface. Referring to FIG. 8b, by taking the center of the stent 11 as the vertex, the angle (represented by β in the figure) between the two first connecting sub-parts 143a may be greater than or equal to 120° and less than or equal to 240°. Further, β may be greater than or equal to 130° and less than or equal to 230°. Further, β may be greater than or equal to 110° and less than or equal to 220°. For example, β may be 120°, 125°, 130°, 140°, 160°, 200°, 210°, 220°, 230°, 235°, 240°, or the like.

In some embodiments, referring to FIG. 8a, there may be three first connecting sub-parts 143a. The three first connecting sub-parts 143a may all be point-like from the top-view surface. By taking the center of the stent 11 as the vertex, an angle (represented by α in FIG. 8a) between any two adjacent first connecting sub-parts 143a is 120°. Therefore, the free part 142 swells uniformly, which has a good effect of preventing perivalvular leakage.

It should be noted that the above describes only several specific implementations listed in the embodiments. The embodiments do not impose a specific limitation on the shape and number of the first connecting sub-part 143a, as long as the first connecting sub-part 143a can connect the free part 142 to the outside of the stent 11 to prevent perivalvular leakage. Similarly, the embodiments do not impose a specific limitation on the shapes and numbers of the second connecting sub-part 143b and the extending sub-part 143c (if any), and related specific implementations will not be elaborated.

The extending sub-part 143c may have an extending length. The length of the extending sub-part 143c may be greater than 0 and less than or equal to the nominal length of the stent 11. For example, when there is one extending sub-part 143c of the same connecting part 143, the above length is a length of the single extending sub-part 143c. When there is more than one extending sub-part 143c of the same connecting part 143, the above length is a total length of the single extending sub-parts 143c. When the lengths of the extending sub-parts 143c are within the above ranges, the free part 142 are relatively loosely connected to the outside of the stent 11, so that the radial extension of the free part 142 after swelling is proper, and an axial margin for the free part 142 with the contraction of the stent 11 is proper too. When there are at least two connecting parts 143, the above length may be a total length of the extending sub-parts 143c of the different connecting parts 143.

Further, the length of each extending sub-part 143c may be 0.5% to 90%, 1% to 80%, 5% to 80%, 8% to 50%, 10% to 30%, 10% to 40%, 20% to 30%, 40% to 50%, 30% to 70%, 50% to 60%, 60% to 85%, or 70% to 90% of the nominal length of the stent 11. For example, the length of each extending sub-part 143c may be 0.5%, 1%, 5%, 8%, 10%, 20%, 30%, 40%, 45%, 50%, 60%, 70%, 80%, 85%, 90%, or 100% of the nominal length of the stent 11. Exemplarily, the length of each extending sub-part 143c may be 0.5 mm, 1 mm, 5 mm, 8 mm, 10 mm, 20 mm, 30 mm, 40 mm, 45 mm, 50 mm, 60 mm, or any range within the above data.

Referring to FIG. 7, in some embodiments, further, the free part 142 may include a swelling sub-part 142b and a blood-permeable sub-part 142c which are connected to each other. The swelling sub-part 142b may be connected to the closing part 141. The swelling sub-part 142b may swell in the direction away from the stent 11 under the pressure of the backflow blood to abut against the surrounding tissues 2 and achieve sealing. The blood-permeable sub-part 142c can allow blood to permeate through, so that blood (if any) between the blood-permeable sub-part 142c and the surrounding tissues 2 can enter the swelling sub-part 142b via the blood-permeable sub-part 142c. The swelling sub-part 142b and the blood-permeable sub-part 142c may be integrally formed, or may be detachably connected (for example, sutured, glued, and/or welded).

The blood-permeable sub-part 142c may be connected to the first connecting sub-part 143a of the connecting part 143. It can be understood that the connecting part 143 may hinder the swelling of the swelling sub-part 142b, and the blood-permeable sub-part 142c connected to the first connecting sub-part 143a can increase the radial extension of the swelling sub-part 142b. Also, the swelling sub-part 142b may be connected to the first connecting sub-part 143a too.

An axial length of the blood-permeable sub-part 142c may be greater than 0 and less than or equal to 40% of the nominal length of the stent 11. In this way, when the blood (if any) between the blood-permeable sub-part 142c and the surrounding tissues 2 enters the swelling sub-part 142b through the blood-permeable sub-part 142c, the blood in the swelling sub-part 142b may not leak out from the blood-permeable sub-part 142c, thus achieve a good effect of preventing perivalvular leakage. When the axial length of the blood-permeable sub-part 142c is greater than 40% of the nominal length of the stent 11, the swelling sub-part 142b may have an insufficient accommodating space, and the blood inside may leak out through the blood-permeable sub-part 142c. Further, the axial length of the blood-permeable sub-part 142c may be 0.5% to 35%, 1% to 40%, 5% to 35%, 1% to 25%, 1% to 20%, 8% to 15%, 10% to 20%, 10% to 25%, or 15% to 30% of the nominal length of the stent 11. For example, the axial length of the blood-permeable sub-part 142c may be 0.5%, 1%, 5%, 8%, 10%, 15%, 20%, 25%, 30%, 35%, or 40% of the nominal length of the stent 11. Exemplarily, the length of the blood-permeable sub-part 142c may be 0.3 mm, 0.5 mm, 1 mm, 3 mm, 5 mm, 8 mm, 10 mm, 15 mm, 18 mm, 20 mm, or any range between the above data.

The blood-permeable sub-part 142c may be made from a blood-permeable material (such as a high-permeability polymer film material). Referring to FIG. 7, the blood-permeable sub-part 142c may be provided with an opening 142d for allowing blood to pass through. The opening 142d may be triangular, circular, square, polygonal, waist-shaped, irregularly shaped, or the like. There may be one or at least two openings 142d. The at least two openings 142d may be spaced apart or connected in the circumferential direction of the stent 11.

The outer skirt 14 may be made from at least one of a biocompatible polymer film, a natural biological tissue, or a surface-modified biocompatible material. The biocompatible polymer film may be at least one of polyurethane, polytetrafluoroethylene, expanded polytetrafluoroethylene, polylactic acid, L-polylactic acid, D-polylactic acid, polyhydroxyacetic acid, polycaprolactone, polyamide, polyethylene terephthalate, poly(galactose), or a copolymer of lactide/caprolactone. The natural biological tissue may be decellularized bovine pericardium, porcine pericardium, porcine aortic valve, fish maw, a small intestine tissue of a mammal, or the like. The surface-modified biocompatible material may be at least one of polyurethane, polyethylene terephthalate, polyethylene, polypropylene, polytetrafluoroethylene, polydimethylsiloxane, polymethyl methacrylate, or a polyoxymethylene material. At least two of the closing part 141, the free part 142, and the connecting part 143 may be made of the same material or different materials.

A spread shape of the outer skirt 14 may include at least one of a triangle, a rectangle, a polygon, a semicircle, a semi-ellipse, a sector, or an irregular shape. For example, a spread shape of the extending end 142a of the free part 142 may be roughly like a straight line, a curved line, a broken line, or the like. For example, referring to FIG. 6, in an embodiment, the shape of the extending end 142a may be consistent with the shape of the outflow end 112, and the extending end 142a may be sawtooth-shaped. The outer skirt 14 includes a plurality of connecting parts 143. The axial distance between some connecting parts 143 and the extending end 142a of the free part 142 is 0. For example, as shown in FIG. 6, the extending end 142a of the free part 142 may be connected to the outflow end 112 of the stent 11 by a suture or in another way, and the connecting parts 143 of the outer skirt 14 essentially corresponding to the suture essentially overlap the extending end 142a. The axial distance between the other connecting parts 143 included in the outer skirt 14 and the extending end 142a of the free part 142 is greater than 0 mm and less than 10 mm, such as the two connecting parts 143 arranged in the axial direction below the outflow end 112 shown in FIG. 6. The connecting parts 143 arranged at a plurality of positions at different axial distances from the extending end 142a can balance the effective utilization rate of the free part 142 and the stability of connecting the outer skirt 14 to the stent 11 or the inner skirt 13. Referring to FIG. 1a to FIG. 5 and FIG. 7, in some other embodiments, the spread shape of the extending end 142a may be like a straight line. The spread shape of the closing part 141 may be roughly like a straight line, a curved line, a broken line, or the like.

### Embodiment I

Referring to FIG. 1a to FIG. 1d, an artificial heart valve 100 includes a stent 11, a leaflet assembly 12, an inner skirt 13, and an outer skirt 14. The stent 11 has an inflow end 111 and an outflow end 112. A plurality of stent rods 114 of the stent 11 are arranged in a crossed manner to form a plurality of stent holes 113. The leaflet assembly 12 is arranged in the stent 11. The leaflet assembly 12 has an inflow edge 121 facing the outflow end 112, and includes three leaflets arranged in the stent 11 in a circumferential direction of the stent 11.

The inner skirt 13 is arranged on an inner surface of the stent 11. An inflow skirt edge 131 of the inner skirt 13 is arranged close to the inflow end 111. An outflow skirt edge 132 is connected to the inflow edge 121 of the leaflet assembly 12. The outflow skirt edge 132 is approximately flush with the inflow edge 121, and an axial distance between the outflow skirt edge 132 and the inflow end 111 is slightly greater than an axial distance between the inflow edge 121 and the inflow end 111.

The outer skirt 14 is arranged at an outer periphery of the stent 11. The outer skirt 14 includes a closing part 141, a free part 142, and a connecting part 143. The closing part 141 is arranged outside the stent 11, and the closing part 141 is connected to the inflow skirt edge 131 of the inner skirt 13. The free part 142 is connected to the closing part 141 and extends in a direction away from the inflow end 111. An extending end 142a of the free part 142 is located between the inflow edge 121 and the outflow end 112. A plurality of first connecting sub-parts 143a of the connecting part 143 are connected to the extending end 142a of the free part 142 and are point-like, and the first connecting sub-parts 143a are spaced apart in a circumferential direction of the free part 142. The stent holes 113 located between the outflow end 112 and the inflow edge 121 are exposed to enable backflow blood to pass through and flow into the free part 142.

### Embodiment II

Structures that are the same as those in Embodiment I will not be elaborated in Embodiment II. Referring to FIG. 2, the inflow skirt edge 131 is located between the inflow edge 121 and the extending end 142a, and the axial distance between the outflow skirt edge 132 and the inflow end 111 is significantly greater than the axial distance between the inflow edge 121 and the inflow end 111.

### Embodiment III

Structures that are the same as those in Embodiment I and Embodiment II will not be elaborated in Embodiment III. Referring to FIG. 3, the inner skirt 13 is provided with a via hole 133 in a portion located between the inflow edge 121 and the inflow skirt edge 131, so that the backflow blood can pass through and flow into the free part 142.

### Embodiment IV

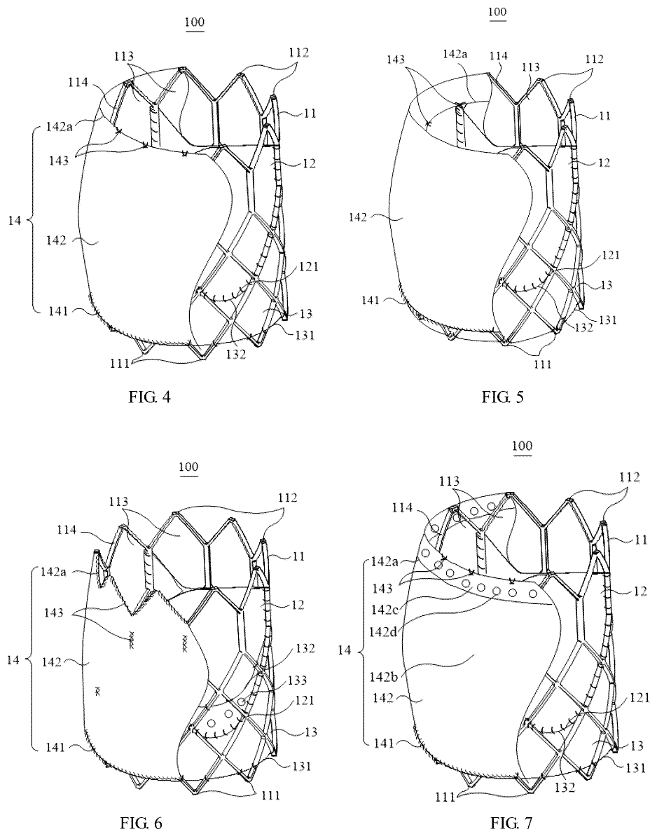

Structures that are the same as those in Embodiment I to Embodiment III will not be elaborated in Embodiment IV. Referring to FIG. 4, the extending end 142a of the free part 142 extends to the outflow end 112.

### Embodiment V

Structures that are the same as those in Embodiment I to Embodiment IV will not be elaborated in Embodiment V. Referring to FIG. 5, the inflow skirt edge 131 of the inner skirt 13 is located between the closing part 141 and the inflow end 111, and the axial distance between the inflow skirt edge 131 and the inflow end 111 is greater than the axial distance between the closing part 141 and the inflow end 111. The extending end 142a of the free part 142 extends out of the outflow end 112 and is folded back into the stent 11.

### Embodiment VI

Structures that are the same as those in Embodiment I to Embodiment V will not be elaborated in Embodiment VI. Referring to FIG. 6, the free part 142 extends to the outflow end 112, and the extending end 142a is sawtooth-shaped. There are a plurality of connecting parts 143. The first connecting sub-part 143a of one connecting part 143 is ringlike and connected to the extending end 142a, and the first connecting sub-parts 143a of the remaining connecting parts 143 are strip-shaped and are spaced apart close the extending end 142a.

### Embodiment VII

Structures that are the same as those in Embodiment I to Embodiment VI will not be elaborated in Embodiment VII. Referring to FIG. 7, the free part 142 includes a swelling sub-part 142b and a blood-permeable sub-part 142c. The blood-permeable sub-part 142c is provided with an opening 142d that allows blood to pass through. The blood-permeable sub-part 142c extends to the outflow end 112, and the connecting part 143 is connected to an end of the blood-permeable sub-part 142c close to the outflow end 112.

### Embodiment VIII

Structures that are the same as those in Embodiment I to Embodiment VII will not be elaborated in Embodiment VIII. Referring to FIG. 8a, there are three connecting parts 143, namely a first connecting part 41, a second connecting part 42, and a third connecting part 43. The first connecting part 41, the second connecting part 42, and the third connecting part 43 are spaced apart in the circumferential direction of the stent 11. By taking a center of the stent 11 as a vertex, an angle (represented by a in the figure) between any two adjacent first connecting sub-parts from a top-view surface is 120°.

### Embodiment IX

Structures that are the same as those in Embodiment I to Embodiment VIII will not be elaborated in Embodiment IX. Referring to FIG. 8b, there are two connecting parts 143, namely a fourth connecting part 44 and a fifth connecting part 45. The fourth connecting part 44 and the fifth connecting part 45 are spaced apart in the circumferential direction of the stent 11. The fourth connecting part 44 and the fifth connecting part 45 are both strip-shaped (rodlike). By taking a center of the stent 11 as a vertex, an angle (represented by β in the figure) between the first connecting sub-parts 143a of the two connecting parts 143 from a top-view surface is greater than 120° and less than 180°.

### Embodiment X

Structures that are the same as those in Embodiment I to Embodiment IX will not be elaborated in Embodiment X. Referring to FIG. 8c, there are five connecting parts 143, namely a sixth connecting part 46, a seventh connecting part 47, an eighth connecting part 48, a ninth connecting part 49, and a tenth connecting part 410. The sixth connecting part 46 includes a first connecting sub-part 143a, two second connecting sub-parts 143b, and two extending sub-parts 143c. The two extending sub-parts 143c are inclined radially relative to each other and are intersected at the first connecting sub-part 143a. The two second connecting sub-parts 143b may be regarded as a head end and a tail end of the sixth connecting part 46, namely, a middle part of the sixth connecting part 46 is connected to the free part 142, and the two ends are connected to the stent 11. The seventh connecting part 47 includes two first connecting sub-parts 143a, one second connecting sub-part 143b, and two extending sub-parts 143c. Namely, two ends of the seventh connecting part 47 are connected to the free part 142, and a middle part is connected to the stent 11. The extending sub-parts (middle parts) of the eighth connecting part 48, the ninth connecting part 49, and the tenth connecting part 410 are connected to each other.

### Embodiment XI

Structures that are the same as those in Embodiment I to Embodiment X will not be elaborated in Embodiment XI. Referring to FIG. 8d, there are three connecting parts 143, namely an eleventh connecting part 411, a twelfth connecting part 413, and a thirteenth connecting part 412. The eleventh connecting part 411 includes a plurality of first connecting sub-parts, a plurality of second connecting sub-parts, and a plurality of extending sub-parts. Two ends of the plurality of extending sub-parts are respectively connected to the first connecting sub-parts and the second connecting sub-parts. The twelfth connecting part 413 includes a plurality of first connecting sub-parts, a plurality of second connecting sub-parts, and a plurality of extending sub-parts. Two ends of one extending sub-part are connected to the second connecting sub-parts, and two ends of the remaining extending sub-parts are respectively connected to the first connecting sub-parts and the second connecting sub-parts. The thirteenth connecting part 412 is ringlike from a top-view surface. The thirteenth connecting part 412 includes one first connecting sub-part, one second connecting sub-part, and two extending sub-parts. Two ends of the two extending sub-parts are respectively connected to the same first connecting sub-part and the same second connecting sub-part.

### Embodiment XII

Parts that are the same as those in Embodiment I to Embodiment XI will not be elaborated in Embodiment XII. Referring to FIG. 10d, the second connecting sub-part is connected to the outflow skirt edge 132 of the inner skirt 13.

The solutions in FIG. 10a to FIG. 10c are the same as some of the solutions in Embodiment I to Embodiment XII, and will not be elaborated here.

The foregoing descriptions are merely specific implementations of the embodiments, but are not intended to limit their scope. Any variation or replacement easily figured out by a person skilled in the art shall fall within the scope of the embodiments.

1.-18. (canceled)

19. An artificial heart valve, comprising:
a stent comprising an inflow end and an outflow end, wherein a plurality of stent holes are formed in the stent;
a leaflet assembly arranged in the stent, wherein the leaflet assembly has an inflow edge facing the outflow end;
an inner skirt arranged on an inner surface of the stent, wherein the inner skirt comprises an inflow skirt edge close to the inflow end and an outflow skirt edge close to the outflow end, and the inner skirt is connected to the inflow edge; and
an outer skirt, at least partially arranged at an outer periphery of the stent, wherein the outer skirt comprises a closing part, a free part, and a connecting part; the closing part is arranged outside the stent and is close to the inflow end; the closing part is connected to the inner skirt;
the free part is connected to the closing part and extends in a direction away from the inflow end; the connecting part is connected to a portion of the free part close to the outflow end, and the connecting part is connected to the stent and/or the inner skirt; backflow blood flows through the stent holes located between the outflow end and the inflow edge and flows into the free part; the free part swells in a direction away from the stent under pressure of the backflow blood to abut against surrounding tissues and achieve sealing.

20. The artificial heart valve according to claim 19, wherein an axial distance between the closing part and the inflow end is A1; a minimum axial distance between the inflow edge and the inflow end is B1; an axial distance between the inflow skirt edge and the inflow end is C1; an axial distance between the outflow skirt edge and the inflow end is C2; and
$$A 1 \leq B 1 , C 1 < A 1 , C 2 > B 1 ; or ,$$
$$A 1 > B 1 , C 1 < B 1 , C 2 > A 1 .$$

21. The artificial heart valve according to claim 20, wherein A1 is greater than or equal to 1% of a nominal length of the stent and less than or equal to 49% of the nominal length of the stent.

22. The artificial heart valve according to claim 20, wherein an axial distance between an extending end of the free part after swelling and the inflow end is A2, and a difference between A2 and B1 is greater than or equal to 30% of a nominal length of the stent and less than or equal to 74% of the nominal length of the stent.

23. The artificial heart valve according to claim 19, wherein a radial extension of the free part after swelling is greater than or equal to 5% of a nominal diameter of the stent and less than or equal to half of a nominal length of the stent.

24. The artificial heart valve according to claim 19, wherein an axial distance between
an extending end of the free part after swelling and the closing part is L;
an axial distance between the outflow end and the closing part is H; and
a length of the free part is greater than or equal to L and less than or equal to (H−1/2L)π.

25. The artificial heart valve according to claim 19, wherein a thickness of the free part is greater than or equal to 5 μm and less than or equal to 200 μm.

26. The artificial heart valve according to claim 19, wherein the connecting part comprises a first connecting sub-part and a second connecting sub-part which are connected to each other; or
the connecting part comprises a first connecting sub-part, a second connecting sub-part, and an extending sub-part, and two ends of the extending sub-part are respectively connected to the first connecting sub-part and the second connecting sub-part; and
the first connecting sub-part is connected to the free part; and the second connecting sub-part is connected to the stent and/or the inner skirt.

27. The artificial heart valve according to claim 26, wherein there are a plurality of connecting parts; the plurality of connecting parts are arranged in a spacing manner in a circumferential direction of the free part; or, in the same connecting part, there are a plurality of first connecting sub-parts.

28. The artificial heart valve according to claim 27, wherein an axial distance between at least one of the first connecting sub-parts and an extending end of the free part is greater than or equal to 0 and less than or equal to 10 mm.

29. The artificial heart valve according to claim 27, wherein at least one of the first connecting sub-parts is strip-shaped or ringlike from a top-view surface; and
by taking a center of the stent as a vertex, an angle between two ends of the strip-shaped first connecting sub-part from the top-view surface is greater than or equal to 120° and less than 360°; or
at least two adjacent first connecting sub-parts are point-like from the top-view surface; and
by taking the center of the stent as the vertex, an angle between the two first connecting sub-parts from the top-view surface is greater than or equal to 120° and less than or equal to 240°.

30. The artificial heart valve according to claim 26, wherein a length of the extending sub-part is greater than 0 and less than or equal to a nominal length of the stent.

31. The artificial heart valve according to claim 19, wherein the free part comprises a swelling sub-part and a blood-permeable sub-part which are connected to each other; the swelling sub-part is connected to the closing part; the swelling sub-part swells in the direction away from the stent under the pressure of the backflow blood to abut against the surrounding tissues and achieve sealing; the blood-permeable sub-part allows blood to permeate through; and an axial length of the blood-permeable sub-part is greater than 0 and less than or equal to 40% of a nominal length of the stent.

32. The artificial heart valve according to claim 31, wherein the blood-permeable sub-part is made from a blood-permeable material, and/or the blood-permeable sub-part is provided with an opening for allowing blood to permeate through.

33. The artificial heart valve according to claim 19, wherein the outer skirt is made from at least one of a biocompatible polymer film, a natural biological tissue, or a surface-modified biocompatible material.

34. The artificial heart valve according to claim 19, wherein a spread shape of the outer skirt includes at least one of a triangle, a rectangle, a polygon, a semicircle, a semi-ellipse, or a sector.

35. The artificial heart valve according to claim 20, wherein A1≤B1, and the backflow blood permeates through at least a portion of the inner skirt located between the inflow edge and the outflow skirt edge; or
A1>B1, and the backflow blood permeates through at least a portion of the inner skirt located between the closing part and the outflow skirt edge.

36. The artificial heart valve according to claim 20, wherein C1 is greater than 0 and less than or equal to 40% of a nominal length of the stent
